(19) United States
(12) Patent Application Publication
(10) Pub. No.: US 2025/0186134 A1
(43) Pub. Date: Jun. 12, 2025

(54) System and Method for Optic Shape Sensing and Electrical Signal Conduction

(71) Applicant: Bard Access Systems, Inc., Salt Lake City, UT (US)

(72) Inventors: Steffan Sowards, Salt Lake City, UT (US); Anthony K. Misener, Bountiful, UT (US)

(21) Appl. No.: 19/057,193

(22) Filed: Feb. 19, 2025

Related U.S. Application Data
(63) Continuation of application No. 17/191,551, filed on Mar. 3, 2021, now Pat. No. 12,232,818.
(60) Provisional application No. 62/984,552, filed on Mar. 3, 2020.

(51) Int. Cl. A61B 34/20; A61B 5/06; A61B 5/318; G02B 6/02; G02B 6/12; G02B 6/42

(52) U.S. Cl. CPC A61B 34/20; A61B 5/065; A61B 5/318; G02B 6/02042; G02B 6/02057; A61B 2034/2061; A61B 2562/0266; G02B 2006/12138; G02B 6/4293

(57) ABSTRACT

A medical device operating as a stylet is described. The medical device can include an insulating layer (or sheath) encapsulating both a multi-core optical fiber and a conductive medium. The optical fiber can include a cladding and a plurality of core fibers spatially arranged within the cladding. Each of the core fibers can include a plurality of sensors distributed along a longitudinal length of that corresponding core fiber and each of these sensors can be configured to: (i) reflect a light signal of a different spectral width based on received incident light, and (ii) change a characteristic of the reflected light signal for use in determining a physical state of the multi-core optical fiber. The conductive medium can provide a pathway for electrical signals detected at a distal portion of the conductive medium. The conductive medium may be concentric to the cladding, but separate and adjust thereto.

### PRIORITY

This application is a continuation of U.S. patent application Ser. No. 17/191,551, filed Mar. 3, 2021, now U.S. Pat. No. 12,232,818, which claims the benefit of priority to U.S. Provisional Application No. 62/984,552, filed Mar. 3, 2020, each of which is incorporated by reference in its entirety into this application.

### BACKGROUND

In the past, certain intravascular guidance of medical devices, such as guidewires and catheters for example, have used fluoroscopic methods for tracking tips of the medical devices and determining whether the tips are appropriately localized in their target anatomical structures. However, such fluoroscopic methods expose patients and their attending clinicians to harmful X-ray radiation. Moreover, in some cases, the patients are exposed to potentially harmful contrast media needed for the fluoroscopic methods.

More recently, electromagnetic tracking systems have been used involving multimodal stylets. Generally, electromagnetic tracking systems feature three components: a field generator, a sensor unit and control unit. The field generator uses several coils to generate a position-varying magnetic field, which is used to establish a coordinate space. Attached to the stylet, such as near a distal end (tip) of the stylet for example, the sensor unit includes small coils in which current is induced via the magnetic field. Based on the electrical properties of each coil, the position and orientation of the medical device may be determined within the coordinate space. The control unit controls the field generator and captures data from the sensor unit.

Although electromagnetic tracking systems avoid line-of-sight reliance in tracking the tip of a stylet while obviating radiation exposure and potentially harmful contrast media associated with fluoroscopic methods, electromagnetic tracking systems are prone to interference. More specifically, since electromagnetic tracking systems depend on the measurement of magnetic fields produced by the field generator, these systems are subject to electromagnetic field interference, which may be caused by the presence of many different types of consumer electronics such as cellular telephones.

Additionally, electromagnetic tracking systems are subject to signal drop out, depend on an external sensor, and are defined to a limited depth range.

Disclosed herein is a fiber optic shape sensing system and methods thereof, which is not subject to the disadvantages associated with electromagnetic tracking systems as described above and is capable in its current form of providing confirmation of tip placement or information passed/interpreted as an electrical signal.

### SUMMARY

Briefly summarized, embodiments disclosed herein are directed to a multimodal stylet featuring a multi-core optical fiber and a conductive medium that collectively operate for tracking placement of a catheter or other medical device within a body of a patient. Operating as a medical device, the stylet is configured to return information for use in identifying its physical state (e.g., shape length, shape, form, and/or orientation) of (i) a portion of the stylet (e.g., tip, segment of stylet, etc.) or a portion of a catheter inclusive of at least a portion of the stylet (e.g., tip, segment of catheter, etc.) or (ii) the entire or substantial portion of the stylet or catheter within the body of a patient (hereinafter, described as the “physical state of the stylet” or the “physical state of the catheter”). According to one embodiment of the disclosure, the returned information may be obtained from reflected light signals of different spectral widths, where each reflected light signal corresponds to a portion of broadband incident light propagating along a core of the multi-core optical fiber (hereinafter, “core fiber”) that is reflected back over the core fiber by a particular sensor located on the core fiber. One illustrative example of the returned information may pertain to a change in signal characteristics of the reflected light signal returned from the sensor, where wavelength shift is correlated to (mechanical) strain on the core fiber.

In some embodiments, the stylet includes a multi-core optical fiber, where each core fiber utilizes a plurality of sensors and each sensor is configured to reflect a different spectral range of the incident light (e.g., different light frequency range). Based on the type and degree of strain asserted on the each core fiber, the sensors associated with that core fiber may alter (shift) the wavelength of the reflected light to convey the type and degree of stain on that core fiber at those locations of the stylet occupied by the sensors. The sensors are spatially distributed at various locations of the core fiber between a proximal end and a distal end of the stylet so that shape sensing of the stylet may be conducted based on analytics of the wavelength shifts. Herein, the shape sensing functionality is paired with the ability to simultaneously pass an electrical signal through the same member (stylet) through conductive medium included as part of the stylet.

More specifically, in some embodiments each core fiber of the multi-core optical fiber is configured with an array of sensors, which are spatially distributed over a prescribed length of the core fiber to generally sense external strain those regions of the core fiber occupied by the sensor. Given that each sensor positioned along the same core fiber is configured to reflect light of a different, specific spectral width, the array of sensors enable distributed measurements throughout the prescribed length of the multi-core optical fiber. These distributed measurements may include wavelength shifts having a correlation with strain experienced by the sensor.

According to one embodiment of the disclosure, each sensor may operate as a reflective grating such as a fiber Bragg grating (FBG), namely an intrinsic sensor corresponding to a permanent, periodic refractive index change inscribed into the core fiber. Stated differently, the sensor operates as a light reflective mirror for a specific spectral width (e.g., a specific wavelength or specific range of wavelengths). As a result, as broadband incident light is supplied by an optical light source and propagates through a particular core fiber, upon reaching a first sensor of the distributed array of sensors for that core fiber, light of a prescribed spectral width associated with the first sensor is reflected back to an optical receiver within a console, including a display and the optical light source. The remaining spectrum of the incident light continues propagation through the core fiber toward a distal end of the stylet. The remaining spectrum of the incident light may encounter other sensors from the distributed array of sensors, where each of these sensors is fabricated to reflect light with different specific spectral widths to provide distributed measurements, as described above.

During operation, multiple light reflections (also referred to as “reflected light signals”) are returned to the console from each of the plurality of core fibers of the multi-core optical fiber. Each reflected light signal may be uniquely associated with a different spectral width. Information associated with the reflected light signals may be used to determine a three-dimensional representation of the physical state of the stylet within the body of a patient. Herein, the core fibers are spatially separated with the cladding of the multi-mode optical fiber and each core fiber is configured to separately return light of different spectral widths (e.g., specific light wavelength or a range of light wavelengths) reflected from the distributed array of sensors fabricated in each of the core fibers. A comparison of detected shifts in wavelength of the reflected light returned by a center core fiber (operating as a reference) and the surrounding, periphery core fibers may be used to determine the physical state of the stylet.

More specifically, during vasculature insertion, the clinician may rely on the console to visualize a current physical state (e.g., shape, orientation, etc.) of a catheter guided by the stylet to avoid potential path deviations that would be caused by changes in catheter orientation. As the periphery core fibers reside at spatially different locations within the cladding of the multi-mode optical fiber, changes in orientation (e.g., angular orientation such as bending, etc.) of the stylet imposes different types (e.g., compression or tension) and degrees of strain on each of the periphery core fibers as well as the center core fiber. The different types and/or degree of strain may cause the sensors of the core fibers to apply different wavelength shifts, which can be measured to extrapolate the physical state of the stylet (and catheter).

As an illustrative example, in the area of vascular access, the multimodal stylet would allow for additional information communicated via electrical signal (e.g., an electrocardiogram “ECG”) to be passed via the conductive medium along with shape sensing information via the optical fiber from the stylet. In the case of central access, this would allow for the use of ECG confirmation techniques to be paired with a robust interpretation of the shape and position of the stylet (as well as optical fiber and/or conductive medium). As described in detail below, the multimodal stylet with the multi-core optical fiber and conductive medium may be constructed in accordance with any of a number of configurations, such as a braided configuration, parallel configuration, flex circuit configuration, conductive tubing configuration, or any other configuration selected by taking into account desired stylet rigidity and properties.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 is an illustrative embodiment of a medical device monitoring system;

FIG. 2 is an exemplary embodiment of a stylet assembly of FIG. 1;

FIG. 3 is an embodiment of the stylet for placement within a catheter of FIG. 1;

FIG. 4 is an embodiment of the stylet of FIGS. 1-3 inserted into a vasculature of a patient;

FIG. 5 is an exemplary embodiment of a structure of a section of the multi-core optical fiber included within the stylet 130 of FIGS. 1-3;

FIG. 6A is a first exemplary embodiment of the multimodal stylet of FIG. 1 supporting both an optical and electrical signaling;

FIG. 6B is a cross sectional view of the multimodal stylet of FIG. 6A;

FIG. 7A a second exemplary embodiment of the multimodal stylet of FIG. 1;

FIG. 7B is a first cross sectional view of the multimodal stylet of FIG. 7A;

FIG. 7C is a second cross sectional view of the multimodal stylet of FIG. 7A;

FIG. 8A is a third exemplary embodiment of the multimodal stylet of FIG. 1;

FIG. 8B is a cross sectional view of the multimodal stylet of FIG. 8A;

FIG. 9A is a fourth exemplary embodiment of the multimodal stylet of FIG. 1;

FIG. 9B is a cross sectional view of the multimodal stylet of FIG. 9A;

FIG. 10A a fifth exemplary embodiment of the multimodal stylet of FIG. 1;

FIG. 10B is a cross sectional view of a first embodiment of the multimodal stylet of FIG. 10A with the conductive medium partially encapsulating a portion of the optical fiber;

FIG. 10C is a cross sectional view of a second embodiment of the multimodal stylet of FIG. 10B with the conductive medium partially encapsulating a greater portion of the optical fiber;

FIG. 10D is a cross sectional view of a third embodiment of the multimodal stylet of FIG. 10A with the conductive medium encapsulating a substantial portion of the optical fiber;

FIG. 11A is a sixth exemplary embodiment of the multimodal stylet of FIG. 1; and

FIG. 11B is a cross sectional view of the multimodal stylet of FIG. 11A.

### DETAILED DESCRIPTION

Reference will now be made to figures wherein like structures will be provided with like reference designations. It is understood that the drawings are diagrammatic and schematic representations of exemplary embodiments of the invention, and are neither limiting nor necessarily drawn to scale.

Regarding terms used herein, it should be understood the terms are for the purpose of describing some particular embodiments, and the terms do not limit the scope of the concepts provided herein. Ordinal numbers (e.g., first, second, third, etc.) are generally used to distinguish or identify different components or operations, and do not supply a serial or numerical limitation. For example, “first,” “second,” and “third” components or operations need not necessarily appear in that order, and the particular embodiments including such components or operations need not necessarily be limited to the three components or operations. Similarly, labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The term “logic” is representative of hardware and/or software that is configured to perform one or more functions. As hardware, logic may include circuitry having data processing and/or storage functionality. Examples of such circuitry may include, but are not limited or restricted to a processor, a programmable gate array, a microcontroller, an application specific integrated circuit, combinatorial circuitry, or the like. Alternatively, or in combination with the hardware circuitry described above, the logic may be software in the form of one or more software modules, which may be configured to operate as its counterpart circuitry. The software modules may include, for example, an executable application, a daemon application, an application programming interface (API), a subroutine, a function, a procedure, a routine, source code, or even one or more instructions. The software module(s) may be stored in any type of a suitable non-transitory storage medium, such as a programmable circuit, a semiconductor memory, non-persistent storage such as volatile memory (e.g., any type of random access memory “RAM”), persistent storage such as non-volatile memory (e.g., read-only memory “ROM”, power-backed RAM, flash memory, phase-change memory, etc.), a solid-state drive, hard disk drive, an optical disc drive, or a portable memory device.

For clarity, it is to be understood that the word “proximal” refers to a direction relatively closer to a clinician using the device to be described herein, while the word “distal” refers to a direction relatively further from the clinician. Herein, the “proximal portion” of a stylet disclosed herein, for example, includes a portion of the stylet intended to be near a clinician when the stylet is used on the patient. Likewise, a “proximal end” of the stylet, for example, includes an end intended to be near the clinician when the stylet is in use. The proximal portion of the stylet may include the proximal end of the stylet; however, proximal portion of the stylet does not need to include the proximal end of the stylet.

Similarly, a “distal portion” of the stylet includes a portion of the stylet intended to be near or in a patient when the stylet is used on the patient. Likewise, a “distal end” of the stylet includes an end of the stylet intended to be near or in the patient when the stylet is in use. The distal portion of the stylet can include the distal end of the stylet; however, the distal portion of the stylet does not need include the distal end of the stylet. Also, the words “including,” “has,” and “having,” as used herein, including the claims, shall have the same meaning as the word “comprising.”

Embodiments of the disclosure are generally directed to a multimodal stylet that assists in the placement of a medical device inserted into a body of a patient. An example of such a medical device is a catheter assembly that is inserted into a vein or other vessel of the patient to infuse or aspirate fluids through one or more lumens defined by the catheter for the patient. The system utilizes multi-core optical fiber with reflective gratings in one embodiment to ascertain information regarding the optical fiber during and/or after insertion into the patient's body for rendering a shape and orientation of the stylet.

Unless defined otherwise, all technical and scientific terms used herein have the same meaning as commonly understood by those of ordinary skill in the art.

In one embodiment, the reflective gratings may include fiber Bragg gratings (“FBG”) distributed along a core fiber disposed in/on the stylet (or another probe-like). An outgoing optical signal produced by a light source is incident on each of the FBGs along the core fiber, where each grating reflects light of a prescribed spectral width to produce a return optical signal to the console. According to one embodiment of the disclosure, shifts in wavelength of reflected light signals returned by each of the core fibers may be aggregated based on FBGs associated with the same cross-sectional region of the stylet (or specific spectral width) and a processor of the console may execute shape sensing analytic logic to perform analytics associated with the wavelength shifts (e.g., analysis of degree, comparison between wavelength shifts between periphery core fibers and the center core fiber or between periphery core fibers, etc.) to identify the physical state of the stylet. The data is communicated to a user of the console to identify (and render) its position within the body, two-dimensional (2-D) and three-dimensional (3-D) shape of the stylet along its length, form and shape (e.g., bending, torsion) as well as orientation (including malposition or medical device kinking), etc. Such information is presented by the console to the user in real-time to assist in guiding and placing the medical device (e.g., catheter) along with the stylet as desired within the patient. Additionally, measurements may be made by the console (e.g., ECG signaling through the conductive medium co-existing with the multi-core optical fiber) to ensure proper deployment within the patient. Further details regarding these and other embodiments are given hereafter.

Note that, though the below discussion focuses on usage of the stylet for the placement of a catheter into the body of the patient, the stylet described herein can be employed to place a variety of medical devices, especially other elongate medical devices, in a variety of locations within the patient body. As such, the principles of the present disclosure should not be considered limiting to what is explicitly described herein. Examples of catheter assemblies and medical devices that may benefit from the disclosure may include a peripherally inserted central catheter (“PICC”), central venous catheter (“CVC”), urinary catheter, midline catheter, peripheral catheter, or the like.

In light of the above, the multi-core optical fiber paired with a conductive medium for electrical signal monitoring thus serves multiple modalities. The first modality constitutes an optical modality with shape sensing functionality to determine the physical state of the stylet. The physical state of the stylet provides information to assist a clinician in guiding a catheter assembly to a desired location within the vasculature. The second modality constitutes tip location/navigation system (“TLS”) modality, where the stylet with conductive medium is advanced to detect and avoid any tip malposition during such advancement. Lastly, a third modality constitutes an ECG modality, wherein ECG signal-based catheter tip guidance is employed to enable tracking and guidance of the stylet/catheter tip to a desired position with respect to a node of the patient's heart from which the ECG signals originate.

As an alternative embodiment, the conductive medium may be configured with a channel or groove in which the multi-core optical fiber may reside or one or more core fibers, separate from the multi-core optical fiber, may reside. Also, in lieu of the conductive medium positioned at the distal end of the stylet, it is contemplated that the conductive medium may extend from the console and terminate just distally from the stylet handle, coming into contact with a saline fluid path, while the multi-core optical fiber extends the length of the catheter. Hence, another conductive media (e.g., saline or another conductive fluid through a Luer connectors 340) may provide an electrically conductive path.

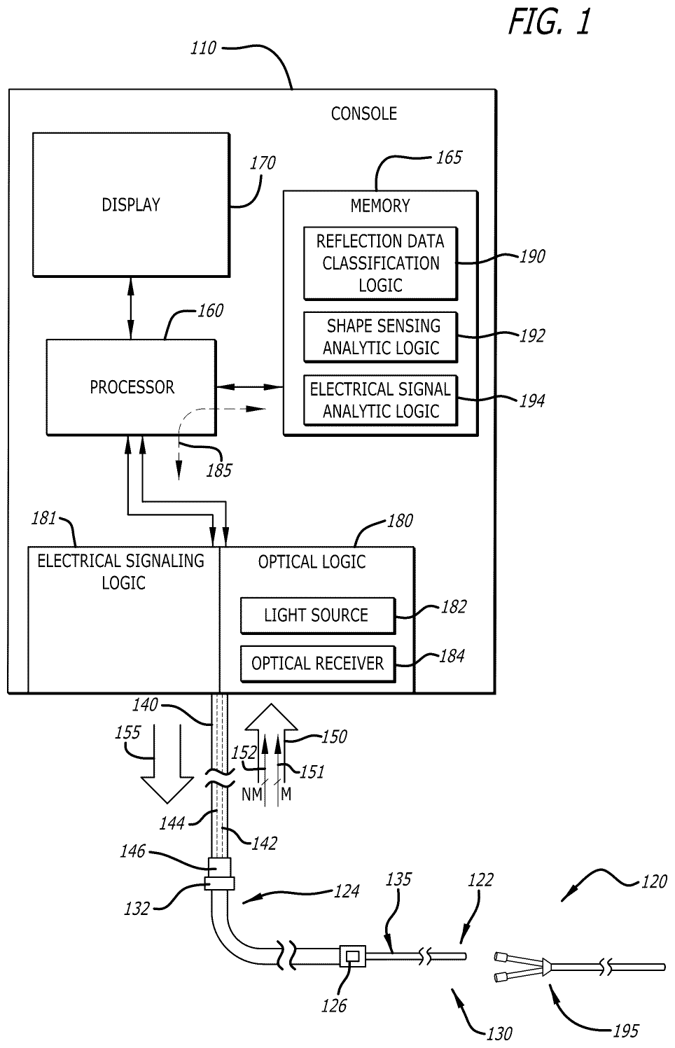

Referring to FIG. 1, an illustrative embodiment of a medical device monitoring system 100 is shown. As shown, the system 100 generally includes a console 110 and a handheld medical device 120 (hereinafter, “stylet assembly”) communicatively coupled to the console 110. For this embodiment, the stylet assembly 120 includes an elongate probe (e.g., stylet) 130 on its distal end 122 and a console connector 132 on its proximal end 124. The console connector 132 enables the stylet assembly 120 to be operably connected to the console 110 via an interconnect 140 including one or more optical fibers 142 (hereinafter, “optical fiber(s)”) and a conductive medium 144 terminated by a single optical/electric connector 146 (or terminated by dual connectors. Herein, the connector 146 is configured to engage (mate) with the console connector 132 to allow for the propagation of light between the console 110 and the stylet assembly 120 as well as the propagation of electrical signals from the stylet 130 to the console 110.

An exemplary implementation of the console 110 includes a processor 160, a memory 165, a display 170 and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Publication No. 2019/0237902, the entire contents of which are incorporated by reference herein. The processor 160, with access to the memory 165 (e.g., non-volatile memory), is included to control functionality of the console 110 during operation. As shown, the display 165 may be a liquid crystal diode (LCD) display integrated into the console 110 and employed as a user interface to display information to the clinician, especially during a catheter placement procedure (e.g., cardiac catheterization). In another embodiment, the display 165 may be separate from the console 110. Although not shown, a user interface is configured to provide user control of the console 110.

For both of these embodiments, the content depicted by the display 165 may change according to which mode the stylet 130 is configured to operate: optical, TLS, ECG, or other modality. In TLS mode, the content rendered by the display 165 may constitute a two-dimensional (2-D) or three-dimensional (3-D) representation of the physical state (e.g., length, shape, form, and/or orientation) of the stylet 130 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below

According to one embodiment of the disclosure, an activation control 126, included on the stylet assembly 120, may be used to set the stylet 130 into a desired operating mode and selectively alter operability the display 165 by the clinician to assist in medical device placement. For example, based on the modality of the stylet 130, the display 165 of the console 110 can be employed for optical modality-based guidance during catheter advancement through the vasculature or TLS modality to determine the physical state (e.g., length, form, shape, orientation, etc.) of the stylet 130. In one embodiment, information from multiple modes, such as optical, TLS or ECG for example, may be displayed concurrently (e.g., at least partially overlapping in time). In one embodiment, the display 165 is a liquid crystal diode (LCD) device.

Referring still to FIG. 1, the optical logic 180 is configured to support operability of the stylet assembly 120 and enable the return of information to the console 110, which may be used to determine the physical state associated with the stylet 130 along with monitored electrical signals such as ECG signaling via an electrical signaling logic 181 that supports receipt and processing of the received electrical signals from the stylet 130 (e.g., ports, analog-to-digital conversion logic, etc.). The physical state of the stylet 130 may be based on changes in characteristics of the reflected light signals 150 received from the stylet 130. The characteristics may include shifts in wavelength caused by strain on certain regions of the core fibers integrated within a multi-core optical fiber 135 positioned within or operating as the stylet 130, as shown below. From information associated with the reflected light signals 150, the console 110 may determine (through computation or extrapolation of the wavelength shifts) the physical state of the stylet 130, notably a catheter 195 configured to receive the stylet 130.

According to one embodiment of the disclosure, as shown in FIG. 1, the optical logic 180 may include a light source 182 and an optical receiver 184. The light source 182 is configured to transmit the broadband incident light 155 for propagation over the optical fiber(s) 142 included in the interconnect 140, which are optically connected to the multi-core optical fiber 135 within the stylet 130. In one embodiment, the light source 182 is a tunable swept laser, although other suitable light source can also be employed in addition to a laser, including semi-coherent light sources, LED light sources, etc.

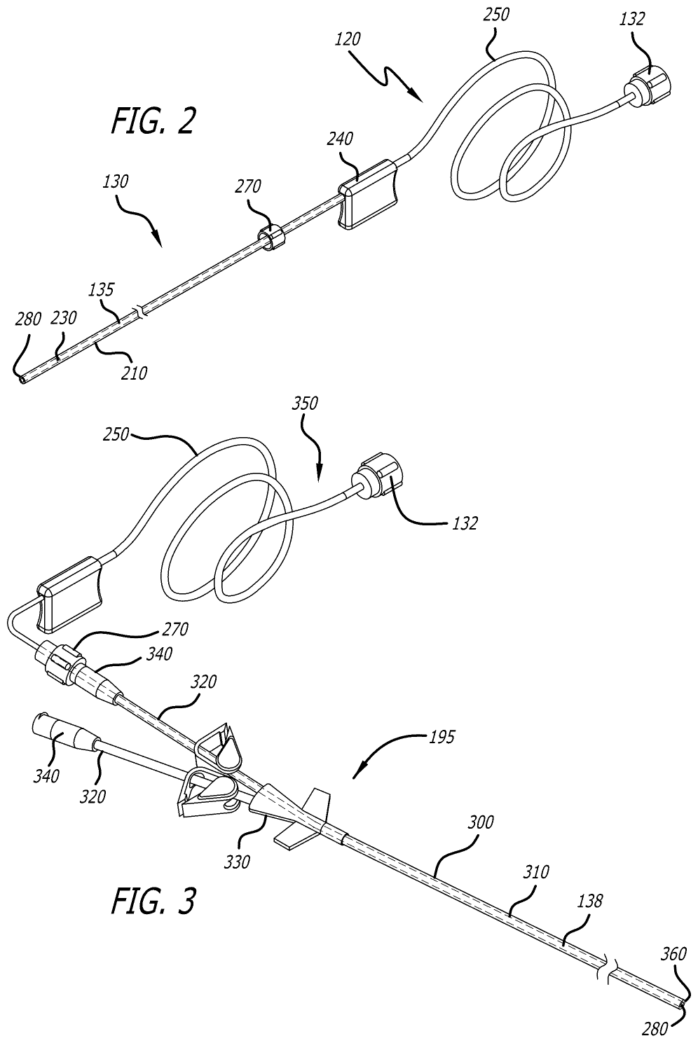

The optical receiver 184 is configured to: (i) receive returned optical signals, namely reflected light signals 150 received from optical fiber-based reflective gratings (sensors) fabricated within each core fiber of the multi-core optical fiber 135 deployed within the stylet 130 (see FIG. 2), and (ii) translate the reflected light signals 150 into reflection data 185, namely data in the form of electrical signals representative of the reflected light signals including wavelength shifts caused by strain. The reflected light signals 150 associated with different spectral widths may include reflected light signals 151 provided from sensors positioned in the center core fiber (reference) of the multi-core optical fiber 135 and reflected light signals 152 provided from sensors positioned in the periphery core fibers of the multi-core optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the processor 160, which governs their operation. Also, the optical receiver 184 is operably coupled to provide the reflection data 185 to the memory 165 for storage and processing by reflection data classification logic 190. The reflection data classification logic 190 may be configured to: (i) identify which core fibers pertain to which of the received reflection data 185 and (ii) segregate the reflection data 185 provided from reflected light signals 150 pertaining to similar regions of the stylet 130 or spectral widths into analysis groups. The reflection data for each analysis group is made available to shape sensing analytic logic 192 for analytics.

According to one embodiment of the disclosure, the shape sensing analytic logic 192 is configured to compare wavelength shifts measured by sensors deployed in each periphery core fiber at the same measurement region of the stylet 130 (or same spectral width) to the wavelength shift at a center core fiber of the multi-core optical fiber 135 positioned along central axis and operating as a neutral axis of bending. From these analytics, the shape sensing analytic logic 192 may determine the shape the core fibers have taken in 3-D space and may further determine the current physical state of the catheter 195 in 3-D space for rendering on the display 170.

According to one embodiment of the disclosure, the shape sensing analytic logic 192 may generate a rendering of the current physical state of the stylet 130 (and potentially the catheter 195), based on heuristics or run-time analytics. For example, the shape sensing analytic logic 192 may be configured in accordance with machine-learning techniques to access a data store (library) with pre-stored data (e.g., images, etc.) pertaining to different regions of the stylet 130 (or catheter 195) in which reflected light from core fibers have previously experienced similar or identical wavelength shifts. From the pre-stored data, the current physical state of the stylet 130 (or catheter 195) may be rendered. Alternatively, as another example, the shape sensing analytic logic 192 may be configured to determine, during run-time, changes in the physical state of each region of the multi-core optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the stylet 130 (and/or catheter 195), especially to enable guidance of the stylet 130, when positioned at a distal tip of the catheter 195, within the vasculature of the patient and at a desired destination within the body.

The console 110 may further include electrical signal receiver logic 186, which is positioned to receive one or more electrical signals from the stylet 130. The stylet 130 is configured to support both optical connectivity as well as electrical connectivity. The electrical signal receiver logic 186 receives the electrical signals (e.g., ECG signals) from the stylet 130 via the conductive medium 144. The electrical signals may be processed by electrical signal analytic logic 194, executed by the processor 160, to determine ECG waveforms for display.

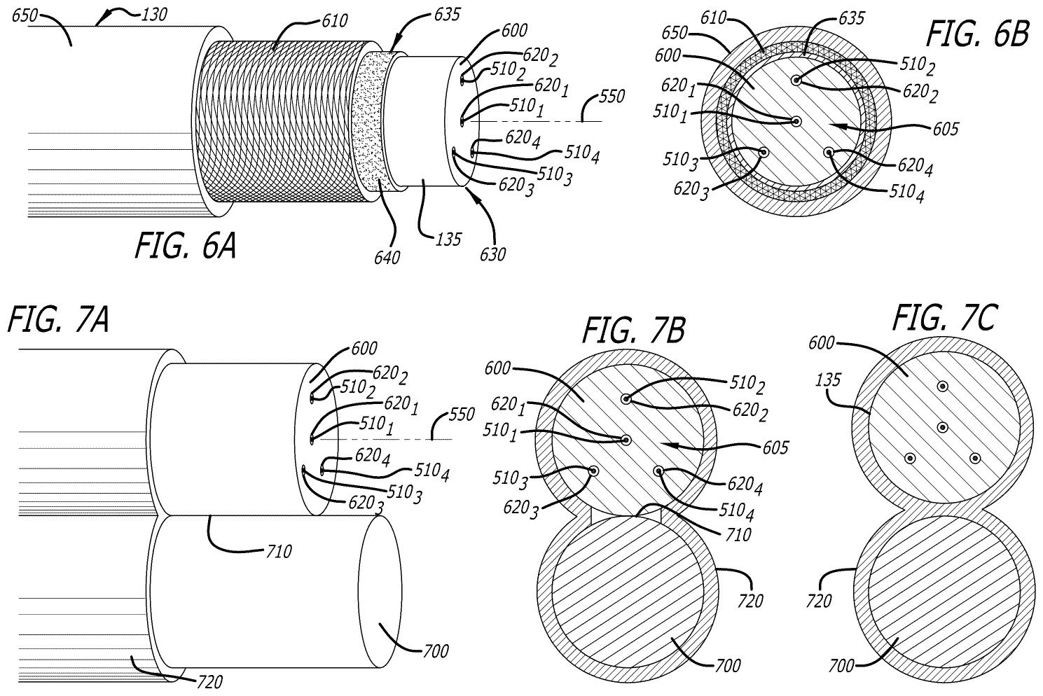

Referring now to FIG. 2, an exemplary embodiment of the stylet assembly 120 to be operably connected to the catheter 195 of FIG. 1 is shown. Herein, the stylet assembly 120 features the stylet 130, which includes an insulating layer 210 encasing a multi-core optical fiber 135 and/or conductive medium 230 as shown in FIGS. 6A-9B and described below. The stylet 130 extends distally from a handle 240 while an interconnect (e.g. tether) 250 extends proximally from the handle 240 and is terminated by the console connector 132 for coupling to the interconnect 140 of the console 110 as shown in FIG. 1. The handle 240 is included with the second interconnect (e.g., tether) 250 to assist with manipulation of the stylet 130 by the user during operation and may be configured to include activation controls 126 as shown in FIG. 1.

As shown, the stylet 130 and the interconnect 250 provide a pathway for outgoing optical signals produced by the light source 182 of the optical logic 180 and returning optical signals, produced by gratings within the core fibers of the multi-core optical fiber 135, for receipt by the photodetector 184 (see FIG. 1). Insulating layers associated with the stylet 130 and the interconnect 250 may vary in density and material to control its rigidity and mechanical properties.

Furthermore, according to one embodiment of the disclosure, the stylet assembly 120 further includes a catheter connector 270, which may be threaded for attachment to a connector of an extension leg of a catheter (see FIG. 3). This connectivity between the connector 270 and a connector of the extension leg connector may be used during the procedure of inserting the stylet 130 into a lumen of the catheter 195, as shown in FIG. 3. When deployed, a distal end of the multi-core optical fiber 135 need not be substantially co-terminal with a distal tip of the catheter. As will be seen, the returned optical signals (reflected light 150) from the sensors (reflective gratings) within each core fiber included with the multi-core optical fiber 135 may be analyzed during its advancement through the patient vasculature.

Note further that, it should appreciated that the term “stylet,” as used herein, can include any one of a variety of devices configured for removable placement within a lumen of the catheter (or other portion of a medical device) to assist in placing a distal end of the catheter in a desired location within the patient's vasculature. Also, note that other connection schemes between the stylet 130 and the console 110 can also be used without limitation.

Referring to FIG. 3, an embodiment of the stylet 130 for placement within the catheter 195 is shown. Herein, the catheter 195 includes an elongate catheter tube 300 defining one or more lumens 310 extending between proximal and distal ends of the catheter tube 300. The catheter tube 300 is in communication with a corresponding extension leg 320 via a bifurcation hub 330. Luer connectors 340 are included on the proximal ends of the extension legs 320.

As shown, the stylet assembly 120 includes the console connector 132 on its proximal end 350 to enable the stylet 130 to operably connect with the console 110 (see FIG. 1). The interconnect 250 distally extends communications from the console 110 to the catheter connector 270, which is configured to threadably engage (or otherwise connect with) the Luer connector 340 of one of the extension legs 320 of the catheter 195. The stylet 130 extends distally from the catheter connector 270 up to a distal-end 280 of the stylet 130. The distal-end 280 of the stylet 130 may be substantially co-terminal with a distal tip 360 of the catheter 195 within the vasculature.

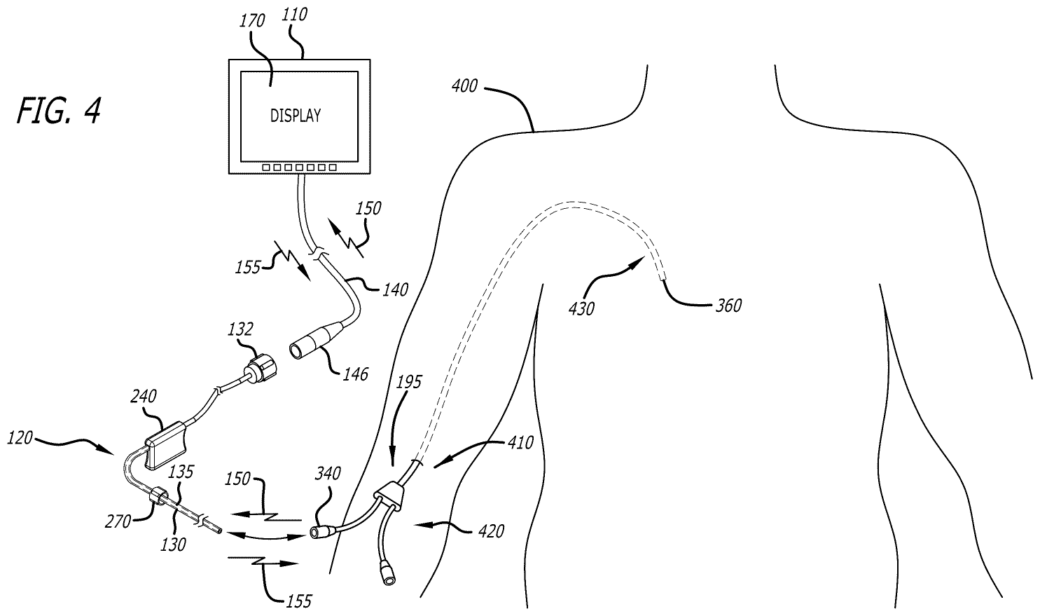

Referring now to FIG. 4, an embodiment of the stylet 130 illustrating its placement within the catheter 195 as the catheter 195 is being inserted into a vasculature of a patient 400 through a skin insertion site 410 is shown. As illustrated in FIG. 4, the catheter 195 generally includes a proximal portion 420 that generally remains exterior to the patient 400 and a distal portion 430 that generally resides within the patient vasculature after placement is complete. The stylet 130 is employed to assist in the positioning of the distal tip 360 of the catheter 195 in a desired position within the patient vasculature. In one embodiment, the desired position for the catheter distal tip 360 is proximate the patient's heart, such as in the lower one-third (⅓^{rd}) portion of the Superior Vena Cava (“SVC”) for this embodiment. Of course, the stylet 130 can be employed to place the catheter distal tip 360 in other locations.

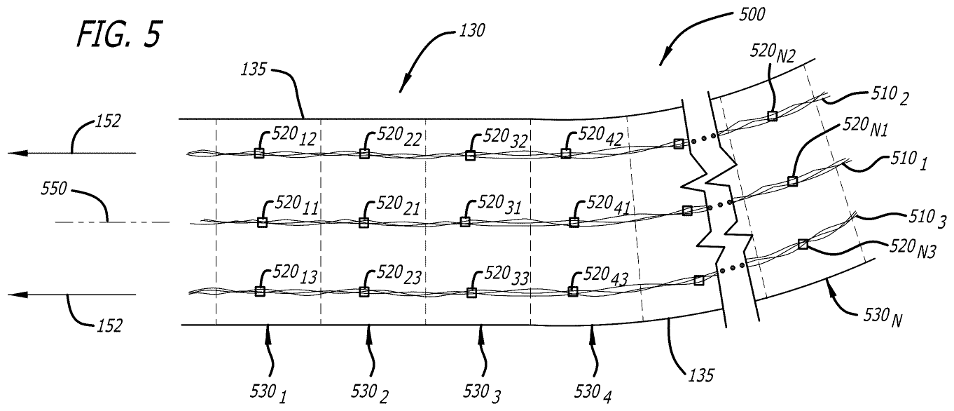

During advancement of the catheter 195, the stylet 130 receives broadband light 155 from the console 110 via interconnect 140, which includes the connector 146 for coupling to the console connector 132 for the stylet assembly 120. The reflected light 150 from sensors (reflective gratings) within each core fiber of the multi-core optical fiber 135 are returned from the stylet 130 over the interconnect 140 for processing by the console 120. The physical state of the stylet 130 may be ascertained based on analytics of the wavelength shifts of the reflected light 150. For example, the strain caused through bending of the stylet 130, and hence angular modification of each core fiber, causes different degrees of deformation. The different degrees of deformation alters the shape of the sensors (reflective grating) positioned on the core fiber, which may cause variations (shifts) in the wavelength of the reflected light from the sensors positioned on each core fiber within the multi-core optical fiber 135, as shown in FIG. 5. From this wavelength shifting, the shape sensing analytic logic 192 within the console 110 (see FIG. 1) may determine the physical state of the stylet 130 (e.g., shape, orientation, etc.).

Referring to FIG. 5, an exemplary embodiment of a right-sided, longitudinal view of a section 500 of the multi-core optical fiber 135 included within the stylet 130 is shown. The multi-core optical fiber section 500 depicts certain core fibers 510₁-510_{M} (M≥2, M=4 as shown) along with the spatial relationship between sensors (e.g., reflective gratings) 520₁₁-520_{NM} (N≥2; M≥2) present within the core fibers 510₁-510_{M}, respectively. As shown, the section 500 is subdivided into a plurality of cross-sectional regions 530₁-530_{N}, where each cross-sectional region 530₁-530_{N} corresponds to reflective gratings 520₁₁-520₁₄ . . . 520_{N1}-520_{N4}. Some or all of the cross-sectional regions 530₁ . . . 530_{N} may be static (e.g., prescribed length) or may be dynamic (e.g., vary in size among the regions 530₁ . . . 530_{N}). A first core fiber 510₁ is positioned substantially along a center (neutral) axis 550 while core fiber 510₂ may be oriented within the cladding of the multi-core optical fiber 130, from a cross-sectional, front-facing perspective, to be position on “top” the first core fiber 510₁. In this deployment, the core fibers 510₃ and 510₄ may be positioned “bottom left” and “bottom right” of the first core fiber 510₁.

Referencing the first core fiber 510₁ as an illustrative example, when the stylet 130 is operative, each of the reflective gratings 520₁-520_{N} reflect light for a different spectral width. As shown, each of the gratings 520₁ᵢ-520_{Ni} (1≤i≤M) is associated with a different, specific spectral width, which would be represented by different center frequencies of f₁ . . . f_{N}, where neighboring spectral widths reflected by neighboring gratings are non-overlapping according to one embodiment of the disclosure.

Herein, positioned in different core fibers 510₂-510₃ but along at the same cross-sectional regions 530-530_{N} of the multi-core optical fiber 135, the gratings 520₁₂-520_{N2} and 520₁₃-520_{N3} are configured to reflect incoming light at same (or substantially similar) center frequency. As a result, the reflected light returns information that allows for a determination of the physical state of the optical fiber 135 (and the stylet 130) based on wavelength shifts measured from the returned, reflected light. In particular, strain (e.g., compression or tension) applied to the multi-core optical fiber 135 (e.g., at least core fibers 510₂-510₃) results in wavelength shifts associated with the returned, reflected light. Based on different locations, the core fibers 510₁-510₄ experience different types and degree of strain based on angular path changes as the stylet 130 advances in the patient.

For example, with respect to the multi-core optical fiber section 500 of FIG. 5, in response to angular (e.g., radial) movement of the stylet 130 is in the left-veering direction, the fourth core fiber 510₄ (see FIG. 6A) of the multi-core optical fiber 135 with the shortest radius during movement (e.g., core fiber closest to a direction of angular change) would exhibit compression (e.g., forces to shorten length). At the same time, the third core fiber 510₃ with the longest radius during movement (e.g., core fiber furthest from the direction of angular change) would exhibit tension (e.g., forces to increase length). As these forces are different and unequal, the reflected light from reflective gratings 520_{N2} and 520_{N3} associated with the core fiber 510₂ and 510₃ will exhibit different changes in wavelength. The differences in wavelength shift of the reflected light signals 152 can be used to extrapolate the physical configuration of the stylet 130 by determining the degrees of wavelength change caused by compression/tension for each of the periphery fibers (e.g., the second core fiber 510₂ and the third core fiber 510₃) in comparison to the wavelength of the reference core fiber (e.g., first core fiber 510₁) located along the neutral axis 550 of the multi-core optical fiber 135. These degrees of wavelength change may be used to extrapolate the physical state of the stylet 130.

Referring now to FIG. 6A, a first exemplary embodiment of the multimodal stylet 130 of FIG. 1 supporting both an optical and electrical signaling is shown. Herein, the stylet 130 features a centrally located multi-core optical fiber 135, which includes a cladding 600 and a plurality of core fibers 510₁-510_{M} (M≥2; M=4) residing within a corresponding plurality of lumens 620₁-620_{M}. While the multi-core optical fiber 135 is illustrated within four (4) core fibers 510₁-510₄, a greater number of core fibers 510₁-510_{M} (M>4) may be deployed to provide a more detailed three-dimensional sensing of the physical state (e.g., shape, orientation, etc.) of the multi-core optical fiber 135 and the stylet 130 deploying the optical fiber 135, a greater number of core fibers 510₁-510_{M} (M>4) may be deployed.

For this embodiment of the disclosure, the multi-core optical fiber 135 is encapsulated within a concentric braided tubing 610 positioned over a low-coefficient of friction layer 635. The braided tubing 610 may feature a “mesh” construction, in which the spacing between the intersecting conductive elements is selected based on the degree of rigidity desired for the stylet 130, as a greater spacing may provide a lesser rigidity, and thereby, a more pliable stylet 130.

According to this embodiment of the disclosure, as shown in FIGS. 6A-6B, the core fibers 510₁-510₄ include (i) a central core fiber 510₁ and (ii) a plurality of periphery core fibers 510₂-510₄, which are maintained within lumens 620₁-620₄ formed in the cladding 600. According to one embodiment of the disclosure, one or more of the lumen 620₁-620₄ may be configured with a diameter sized to be greater than the diameter of the core fibers 510₁-510₄. By avoiding a majority of the surface area of the core fibers 510₁-510₄ from being in direct physical contact with a wall surface of the lumens 620₁-620₄, the wavelength changes to the incident light are caused by angular deviations in the multi-core optical fiber 135 thereby reducing influence of compression and tension forces being applied to the walls of the lumens 620₁-620_{M}, not the core fibers 510₁-510_{M} themselves.

As further shown in FIGS. 6A-6B, the core fibers 510₁-510₄ may include central core fiber 510₁ residing within a central or first lumen 620₁ formed along the first neutral axis 550 and a plurality of core fibers 510₂-510₄ residing within lumens 620₂-620₄ each formed within different areas of the cladding 600 radiating from the first neutral axis 550. In general, the core fibers 510₂-510₄, exclusive of the central core fiber 510₁, may be positioned at different areas within a cross-sectional area 605 of the cladding 600 to provide sufficient separation to enable three-dimensional sensing of the multi-core optical fiber 135 based on changes in wavelength of incident light propagating through the core fibers 510₂-510₄ and reflected back to the console for analysis.

For example, where the cladding 600 features a circular cross-sectional area 605 as shown in FIG. 6B, the core fibers 510₂-510₄ may be positioned substantially equidistant from each other as measured along a perimeter of the cladding 600, such as at “top” (12 o'clock), “bottom-left” (8 o'clock) and “bottom-right” (4 o'clock) locations as shown. Hence, in general terms, the core fibers 510₂-510₄ may be positioned within different segments of the cross-sectional area 605. Where the cross-sectional area 605 of the cladding 600 features a polygon cross-sectional shape (e.g., triangular, square, rectangular, pentagon, hexagon, octagon, etc.), the central core fiber 510₁ may be located at or near a center of the polygon shape, while the remaining core fibers 510₂-510_{M} may be located proximate to angles between intersecting sides of the polygon shape.

Referring still to FIGS. 6A-6B, operating as the conductive medium for the stylet 130, the braided tubing 610 provides mechanical integrity to the multi-core optical fiber 135 and operates as a conductive pathway for electrical signals. For example, the braided tubing 610 may be exposed to a distal tip 630 of the stylet 130. The cladding 600 and the braided tubing 610, which is positioned concentrically surrounding a circumference of the cladding 600, are contained within the same insulating layer 650. The insulating layer 650 may be a sheath or conduit made of protective, insulating (e.g., non-conductive) material that encapsulates both for the cladding 600 and the braided tubing 610, as shown.

For one embodiment of the disclosure, a conductive material such as a conductive epoxy 640 may be affixed to the tip 630, where the braided tubing 610 and/or conductive epoxy 640 may be similarly joined with a termination/connection point created at the proximal portion of the stylet 130. It is contemplated that the handle 240 may include crimping/solder joints to assist in providing an electrical transition (electrical connection) between the braided tubing 610 within the stylet 130 and electrical wires in tether 250 that provides connectivity the interconnect 140 of the console 110. It is contemplated that other electrical connective mechanisms between the tether 250 and the braided tubing 610 may be deployed such as connectivity through abutment of the braided tubing 610 to a conductive element (mesh, gasket, or ring) located at proximal portion (or end) of the stylet 130 (with connectivity to tether 250), through abutment of the braided tubing 610 to conductive tabs/bars integrated into the handle 240 for routing through the tether 250, or through threaded action interface.

Referring now to FIG. 7A, a second exemplary embodiment of the multimodal stylet 130 of FIG. 1 supporting both an optical and electrical signaling is shown. Herein, the stylet 130 features the multi-core optical fiber 135 shown in FIG. 6A, which includes the cladding 600 and the first plurality of core fibers 510₁-510_{M} (M≥3; M=4 for embodiment) residing within the corresponding plurality of lumens 620₁-620_{M}. For this embodiment of the disclosure, the multi-core optical fiber 135 includes the central core fiber 510₁ residing within the central or first lumen 620₁ formed along the first neutral axis 550 and the second plurality of core fibers 510₂-510₄ residing within corresponding lumens 620₂-620₄ positioned in different segments within the cross-sectional area 605 of the cladding 600. Herein, the core fibers 510₁-510_{M} may be deployed in a manner similar to the deployment described above and illustrated in FIGS. 6A-6B.

In contrast of the use of the braided tubing 610 illustrated in FIGS. 6A-6B, the multi-core optical fiber 135 of the multimodal stylet 130 may be paired with a conductive medium 700 such as an electrical wire. According to this embodiment of the disclosure, as shown in FIG. 7A, the conductive medium 700 is configured to propagate electrical (e.g., ECG) signals received by the stylet 130 upon placement of the stylet 130 within a patient. For this embodiment, the conductive medium 700 is positioned without being isolated from the multi-core optical fiber 135, where the conductive medium 700 extends lengthwise adjacent to an outer surface 710 of the cladding 600.

For one embodiment of the disclosure, the handle 240 may include crimping/solder joints to provide electrical connectivity between the conductive medium 700 within electrical wires within the tether 250. It is contemplated that other electrical connective mechanisms to the conductive medium may be deployed as described above.

As illustrated by a cross-sectional view of the stylet 130 in FIGS. 7B-7C, the multi-core optical fiber 135 and the conductive medium 700 may be positioned in parallel, where the optical fiber 135 and the conductive medium 700 are encapsulated within the same insulating layer 720. For this embodiment of the disclosure, the insulating layer 720 operates as a protective casing for both the multi-core optical fiber 135 and the conductive medium 700, where the conductive medium 700 may be in direct physical contact with the cladding 600. As shown in FIG. 7B, the insulating layer 720 operates as a single casing in which the conductive medium 700 is not shielded (insulated) from the cladding 600 of the multi-core optical fiber 135. In contrast, as shown in FIG. 7C, the insulating layer 720 operates as a dual casing in which the conductive medium 700 is shielded (insulated) from the cladding 600 of the multi-core optical fiber 135.

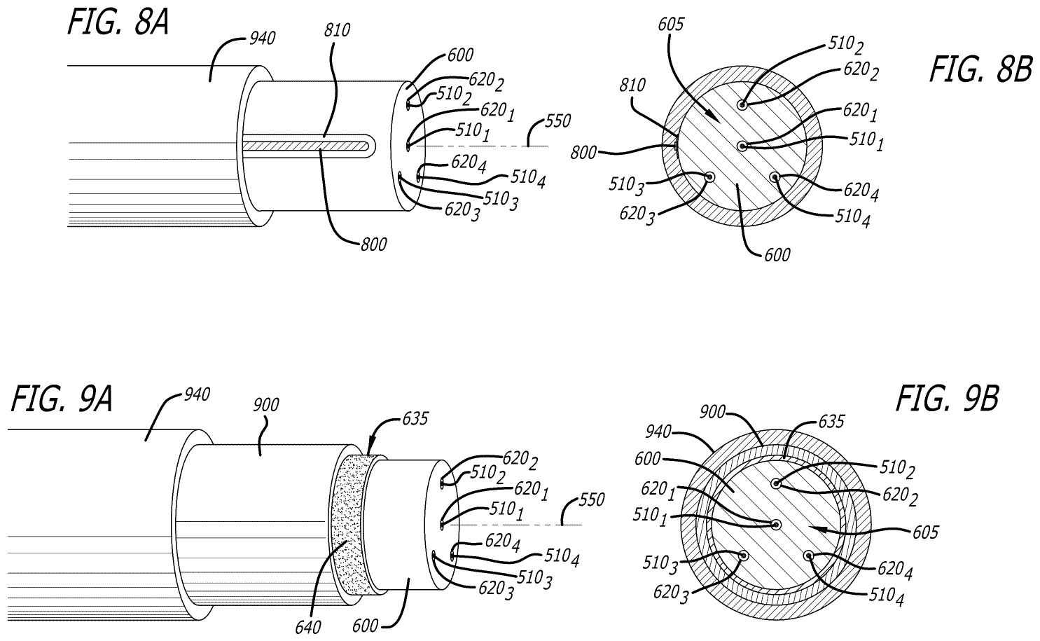

Referring to FIG. 8A, a third exemplary embodiment of the multimodal stylet 130 of FIG. 1 supporting both an optical and electrical signaling is shown. Herein, the multimodal stylet 130 features the multi-core optical fiber 135 shown in FIG. 6A, which includes the cladding 600 and the first plurality of core fibers 510₁-510_{M} (M≥3; M=4 for embodiment) residing within the corresponding plurality of lumens 620₁-620_{M}. For this embodiment of the disclosure, the multi-core optical fiber 135 includes the central core fiber 510₁ residing within the central or first lumen 620₁ formed along the first neutral axis 550 and the second plurality of core fibers 510₂-510₄ residing within corresponding lumens 620₂-620₄ positioned in different segments within the cross-sectional area 605 of the cladding 600. Herein, the multi-core optical fiber 135 may be paired with a flex circuit 800, namely a flexible printed circuit running along a length of the multi-core optical fiber 135.

As shown in FIGS. 8A-8B, the flex circuit 800 is configured to propagate any electrical (e.g., ECG) signals detected by the stylet 130. The flex circuit 800 resides along an outer surface 810 of the cladding 600 and may be encased within an insulating layer (conduit) 820. According to one embodiment of the disclosure, the flex circuit 800 may be attached to the cladding 600 of the multi-core optical fiber 135 through one or more electrical attachment members 820, which may be electrically isolated from the multi-core optical fiber 135. The electrical attachment members 820 may feature a crimping mechanism, one or more solder joints, or the like. For example, the handle 240 of the stylet assembly 120 of FIG. 1, which is attached to the stylet 130, may act as the crimping mechanism to establish connectivity between the flex circuit and electrical wires provided via the tether 250 of the stylet assembly 120.

Referring now to FIG. 9A, a fourth exemplary embodiment of the multimodal stylet 130 of FIG. 1 supporting both an optical and electrical signaling is shown. Herein, the multimodal stylet 130 features the multi-core optical fiber 135 described above and shown in FIG. 6A, which includes the cladding 600 and the first plurality of core fibers 510₁-510_{M} (M≥3; M=4 for embodiment) residing within the corresponding plurality of lumens 620₁-620_{M}. For this embodiment of the disclosure, the multi-core optical fiber 135 includes the central core fiber 510₁ residing within the central or first lumen 620₁ formed along the first neutral axis 550 and the second plurality of core fibers 510₂-510₄ residing within corresponding lumens 620₂-620₄ positioned in different segments within the cross-sectional area 605 of the cladding 600. Herein, the multi-core optical fiber 135 is encapsulated within a conductive tubing 900. The conductive tubing 900 may feature a “hollow” conductive cylindrical member concentrically encapsulating the multi-core optical fiber 135. Such conductive tubing 900 can be formed of a metal or ally including a shape-memory alloy such as nitinol.

Referring to FIGS. 9A-9B, operating as a conductive medium for the stylet 130 in the transfer of electrical signals (e.g., ECG signals) to the console, the conductive tubing 900 may be exposed up to a tip 910 of the stylet 130. For this embodiment of the disclosure, a conductive epoxy 920 (e.g., metal-based epoxy such as a silver epoxy) may be affixed to the tip 910 and similarly joined with a termination/connection point created at a proximal end 930 of the stylet 130. The electrical pathway may be continued through the handle 240 to the connector 132 located on the proximal end 124 of the interconnect 144 of the stylet assembly 120 as shown in FIG. 1. Alternatively, the handle 240 may provide an electrical coupling between the conductive tubing 900 and an electrical wire included as part of the interconnect 144 with the multi-core optical fiber 330. The cladding 600 and the conductive tubing 900, which is positioned concentrically surrounding a circumference of the cladding 600, are contained within the same insulating layer 940. The insulating layer 940 may be a protective conduit encapsulating both for the cladding 600 and the conductive tubing 900, as shown.

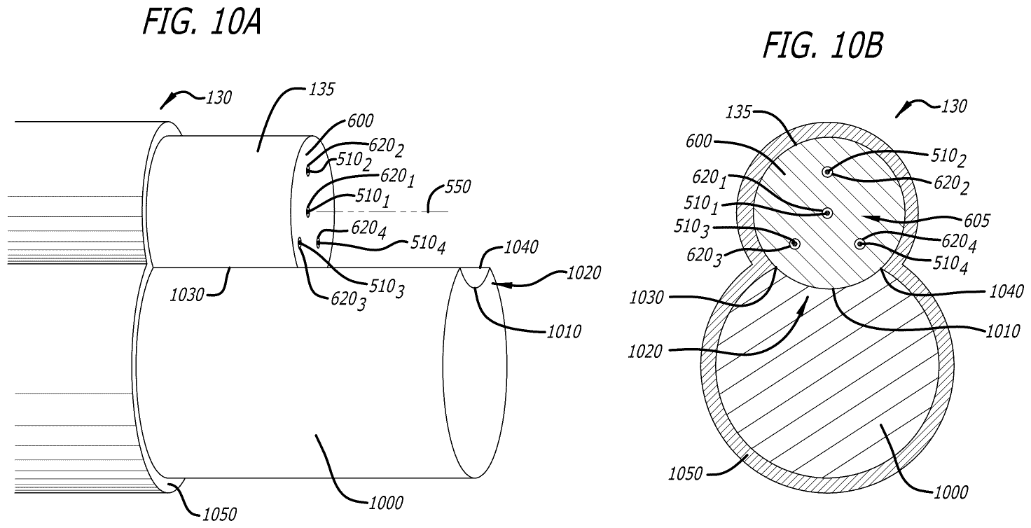

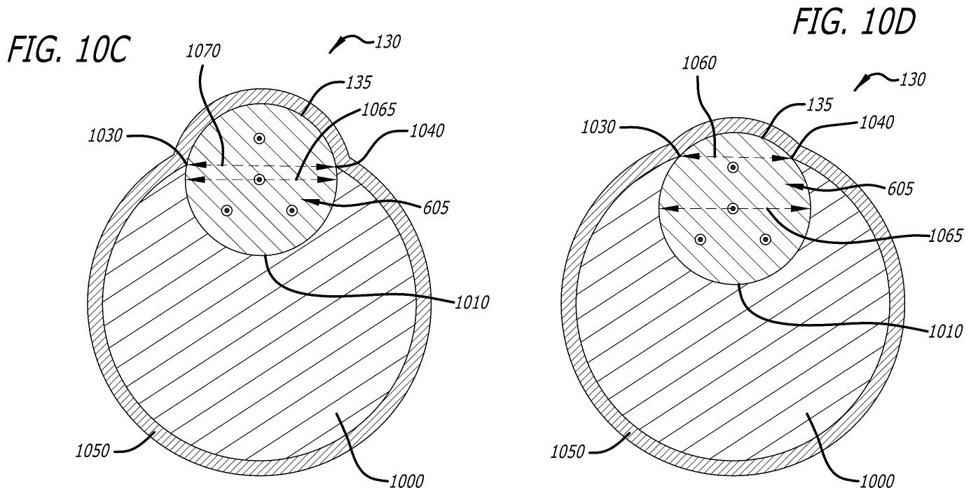

Referring now to FIG. 10A, a fifth exemplary embodiment of the multimodal stylet 130 of FIG. 1 supporting both an optical and electrical signaling is shown. Herein, the stylet 130 features the multi-core optical fiber 135 shown in FIG. 6A, which includes the cladding 600 and the first plurality of core fibers 510₁-510_{M} (M≥3; M=4 for embodiment) residing within the corresponding plurality of lumens 620₁-620_{M}. For this embodiment of the disclosure, the multi-core optical fiber 135 includes the central core fiber 510₁ residing within the central or first lumen 620₁ formed along the first neutral (e.g., central) axis 550 and the second plurality of core fibers 510₂-510₄ residing within corresponding lumens 620₂-620₄ positioned in different segments within the cross-sectional area 605 of the cladding 600. Herein, the core fibers 510₁-510_{M} may be deployed in a manner similar to the deployment described above and illustrated in FIGS. 7A-7C.

In contrast of the paired optical-wire deployment of FIG. 7A, the conductive medium 1000 is contained with a channel 1010 into which a portion of the multi-core optical fiber 135 resides, where the conductive medium 1000 may extend lengthwise and remain adjacent to a segment 1020 of the surface area of the cladding 600. The degree of containment may include the multi-core optical fiber 135 being at least partially encapsulated (see FIG. 10C) or substantially encapsulated (see FIG. 10D) within the channel 1010 of the conductive medium 1000. As shown in FIG. 10C, a portion of the circumference of the multi-core optical fiber 135 (e.g., ranging between forty to sixty percent of the circumference) may be retained within the channel 1010, where a first channel edge 1030 to a second channel edge 1040 may extend slightly beyond a diametrical bisection of the cross-sectional area 605 of the conductive medium 1000. In FIG. 10D, a greater portion of the circumference of the multi-core optical fiber 135 (e.g., greater than sixty percent of the circumference) may be retained within the channel 1010, causing the optical fiber 135 to be “snapped” into the channel 1000 as an opening 1060 of the channel 1010 may be smaller in width than the width of the channel 1065 in which the optical fiber 135 is retained as well as an opening 1070 of the channel 1010 as illustrated in FIG. 10C (but just larger, equal or just smaller than the width of the sheath of the optical fiber as the sheath is made of a flexible material).

As illustrated by a cross-sectional view of a first embodiment of the stylet 130, in FIG. 10B, the multi-core optical fiber 135 and the conductive medium 1000 may be positioned in parallel, where the optical fiber 135 resides within the channel 1010, formed as a recess or grooved portion on the conductive medium (e.g., wire) 1000, and encapsulated within optional insulating layer 1050. For this embodiment of the disclosure, the insulating layer 1050 operates as a protective casing for both the multi-core optical fiber 135 and the conductive medium 1000, where the conductive medium 1000 may be in direct physical contact with the cladding 600 as shown or may be insulated (shielded) from the cladding 600. Cross-sectional views of other embodiments of the stylet 130, with greater encapsulation of the optical fiber 135 within the channel 1010, are shown in FIGS. 10C-10D.

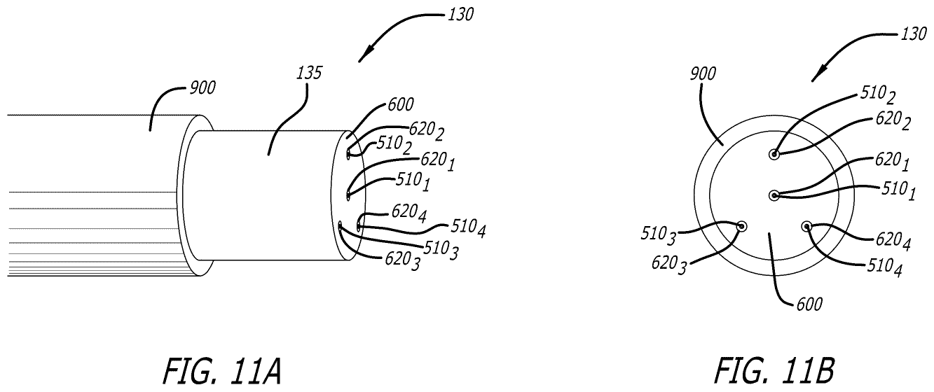

Referring now to FIGS. 11A and 11B, a sixth exemplary embodiment of the multimodal stylet 130 of FIG. 1 supporting both an optical and electrical signaling is shown. Herein, the stylet 130 features the multi-core optical fiber 135 shown in FIG. 6A, which includes the cladding 600 and the first plurality of core fibers 510₁-510_{M} (M≥3; M=4 for embodiment) residing within the corresponding plurality of lumens 620₁-620_{M} as set forth above in the description of the fourth exemplary embodiment of the multimodal stylet 130 of FIGS. 9A and 9B, which description is incorporated herein so as to not burden the disclosure. However, different than the multimodal stylet 130 of FIGS. 9A and 9B, the multi-core optical fiber 135 is encapsulated within only the conductive tubing 900 and optionally affixed thereto with the conductive epoxy 920; the insulating layer 940 shown in FIGS. 9A and 9B is not included. The conductive tubing 900 may feature a “hollow” conductive cylindrical member concentrically encapsulating the multi-core optical fiber 135. Such conductive tubing 900 can be formed of a metal or ally including a shape-memory alloy such as nitinol.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. An optical shape sensing system, comprising:
a console comprising:
(i) a processor,
(ii) a memory, and
(iii) an optical logic including a light source and a photodetector; and
a multimodal stylet optically and electrically coupled to the console, the multimodal stylet comprising:
a conductive medium configured to provide a pathway for electrical signals along a length of the multimodal stylet; and
a multi-core optical fiber in a non-conductive cladding, the multi-core optical fiber comprising:
a plurality of core fibers extending from a proximal end to a distal end of the multi-core optical fiber, the plurality of core fibers comprising a central core fiber extending along a central axis of the multi-core optical fiber and two or more core fibers extending parallel to the central core fiber; and
a plurality of sensors distributed along the multi-core optical fiber from the proximal end to the distal end of the multi-core optical fiber, wherein each of the plurality of sensors is configured to:
reflect a light signal as reflected light with a different spectral width for return to the photodetector based on broadband incident light received from the light source, and
change a characteristic of the reflected light signal for use in determining a physical state of the multi-core optical fiber.

2. The optical shape sensing system according to claim 1, wherein the electrical signals are detected by the conductive medium at a distal end of the multimodal stylet and conveyed along the length of the multimodal stylet to a proximal end of the multimodal stylet.

3. The optical shape sensing system according to claim 1, wherein each core fiber of the plurality of core fibers includes a plurality of sensors distributed along a length thereof.

4. The optical shape sensing system according to claim 1, wherein the physical state of the multi-core optical fiber is selected from the group consisting of a length, a shape, a form, an orientation, and combinations thereof.

5. The optical shape sensing system according to claim 1, wherein the multi-core optical fiber is configured to determine the physical state during insertion of the multimodal stylet into a body of a patient.

6. The optical shape sensing system according to claim 1, wherein each of the plurality of sensors is a reflective grating formed as a permanent, periodic refractive index change inscribed into its corresponding core fiber.

7. The optical shape sensing system according to claim 1, wherein plurality of sensors are positioned at different regions along the multi-core optical fiber.

8. The optical shape sensing system according to claim 1, wherein the multimodal stylet is configured to be removably positioned in a catheter.

9. The optical shape sensing system according to claim 1, wherein the change in the characteristic of the reflected light includes a shift in wavelength applied to the reflected light signal to identify a type of strain.

10. The optical shape sensing system according to claim 1, wherein the electrical signals include an electrocardiogram (ECG) signal.

11. The optical shape sensing system according to claim 1, wherein the multi-core optical fiber is encapsulated within a channel of the conductive medium.

12. The optical shape sensing system according to claim 1, wherein the conductive medium of the multimodal stylet includes a braided tubing.

13. The optical shape sensing system according to claim 12, wherein a distal end of the braided tubing and a distal end of each core fiber of the plurality of core fibers are exposed at a distal end of the multimodal stylet.

14. The optical shape sensing system according to claim 1, wherein the conductive medium of the multimodal stylet includes a conductive tubing positioned concentric to the multi-core optical fiber.

15. The optical shape sensing system according to claim 14, wherein the conductive tubing is nitinol tubing.

16. The optical shape sensing system according to claim 1, wherein the conductive medium of the multimodal stylet includes one or more electrical wires positioned within a first insulating-layer lumen formed by an insulating sheath, and wherein the multi-core optical fiber is positioned within a second insulating-layer lumen formed by the insulating sheath.

17. The optical shape sensing system according to claim 16, wherein the one or more electrical wires and the multi-core optical fiber of the multimodal stylet are electrically isolated by a portion of the first insulating-layer lumen, a portion of the second insulating-layer lumen, or portions of the first insulating-layer lumen and the second insulating-layer lumen.

18. The optical shape sensing system according to claim 1, wherein the conductive medium of the multimodal stylet includes a flexible circuit positioned along an outer surface of the non-conductive cladding and distributed along a length of the multi-core optical fiber.

19. The optical shape sensing system according to claim 1, wherein the multi-core optical fiber is within a circular cross-sectional area defined by the non-conductive cladding, and wherein the two or more core fibers comprise:
a first core fiber positioned at a first arc segment of the circular cross-sectional area in a first radial direction from the central axis of the multi-core optical fiber;
a second core fiber positioned at second arc segment of the circular cross-sectional area in a second radial direction from the central axis of the multi-core optical fiber different from the first radial direction; and
a third core fiber positioned at a third arc segment of the circular cross-sectional area in a third radial direction from the central axis of the multi-core optical fiber different from the first radial direction and the second radial direction.

20. The optical shape sensing system according to claim 19, wherein the first core fiber, the second core fiber, and the third core fiber are positioned such that:
a first obtuse angle is formed between the first core fiber, the central core fiber and the second core fiber;
a second obtuse angle is formed between the first core fiber, the central core fiber and the third core fiber; and
a third obtuse angle is formed between the second core fiber, the central core fiber and the third core fiber.
